(19) United States
(12) Patent Application Publication
(10) Pub. No.: US 2025/0228994 A1
(43) Pub. Date: Jul. 17, 2025

(54) FRAGRANCE LANYARD

(71) Applicant: ADAM ELEMENTS INTERNATIONAL CO., LTD., TAIPEI CITY (TW)

(72) Inventors: CHUNG-YING CHANG, TAIPEI CITY (TW); HUNG-YI LIN, TAIPEI CITY (TW); YING-MIN WANG, TAIPEI CITY (TW)

(21) Appl. No.: 18/820,290

(22) Filed: Aug. 30, 2024

(30) Foreign Application Priority Data
Jan. 17, 2024 (TW) 113200595

(51) Int. Cl. A61L 9/12; A45F 3/00

(52) U.S. Cl. CPC A61L 9/12; A45F 3/00; A45F 2003/002; A61L 2209/13; A61L 2209/14; A61L 2209/15

(57) ABSTRACT

A fragrance lanyard includes a string, a pair of length adjusters, a clasp, a fragrance container, and an aromatic element. The string has an overlapping part, and a hanging ring is formed in a non-overlapping area, each end of the string is wrapped with a string cap, each length adjuster is provided with two lanyard holes such that the cross-section of the length adjuster is designed in an 8-shape for the string to sequentially pass through the pair of length adjusters to adjust the length of the string, the clasp is arranged at the position of the hanging ring, the fragrance container is fixed to any one of the string cap, the length adjuster, or the clasp, and the fragrance container has a containing space provided for installing the aromatic element.

### CROSS-REFERENCE TO RELATED APPLICATIONS

This non-provisional application claims priority under 35 U.S.C. § 119 (a) on Patent Application No(s). 113200595 filed in Taiwan, R.O.C. on Jan. 17, 2024, the entire contents of which are hereby incorporated by reference.

### BACKGROUND OF THE DISCLOSURE

### Technical Field

The present disclosure relates to a fragrance lanyard for hanging various articles, which uses a clasp, a length adjuster or a string cap at the end of a string tail of the lanyard to connect an aromatic element, and the fragrance lanyard is worn on a user's body for emitting fragrance at all time.

### Description of the Related Art

To emphasize personal characteristics or styles, people often wear different decorations to show their personal taste and character. There are many types of decorations, and the common ones include necklaces, rings, earrings, bracelets, pendants, hanging ornaments used for the purpose of matching use to enhance the overall aesthetic looks. In recent years, due to the frequent use of mobile phones, a variety of accessories such as lanyards or finger rings, etc. are used to facilitate the portability and the convenience of use of the mobile phones. This kind of accessories itself does not have too much functionality, and most of the time provides the convenient use and avoids the risk of dropping the mobile phones during use.

At present, most of these accessories are only convenient to hold or easy to use on the back of the mobile phone, in addition to the provision of some decorative means to improve the aesthetic look of the mobile phone, and these decorative means enhances the aesthetic look of the mobile phone through the selected material of the lanyard body or the addition of a pendant, in addition to adding a small bottle of essential oils to a side of the lanyard, so that it can quickly emit fragrance after wearing the accessories However, the lanyard, the hanging ornament or the pendant and the essential oil bottle are still independent items, which will add a lot of burden to the lanyard and is quite inconvenient to use. Obviously, improvements are needed.

In view of the problems above, the present disclosure based on many years of experience in related industry to conduct extensive experiment and research, and finally developed the fragrance lanyard in accordance with the present disclosure's, which is used for hanging various articles such as mobile phones, various cards, and even wallets, so that its clasp, adjuster, or string cap at the end of the string tail can be used to assemble and connect to an aromatic element to be worn on a user's body, in order to continuously emit fragrance, and be removed quickly to refill the source of the fragrance.

### SUMMARY OF THE DISCLOSURE

It is a primary objective of the present disclosure to provide a fragrance lanyard, which includes a string, a pair of length adjusters, a clasp, a fragrance container and an aromatic element. The fragrance container can be quickly installed to any one of the string caps or any one of the length adjusters or the clasp by a fixation means, and the fragrance container has a containing space defined therein and provided for installing the aromatic element in the containing space, so that after the fragrance lanyard is worn on the user's body, it has the effect of emitting fragrance at all time. In addition, the fragrance container can be manufactured directly into any stylish ornament to further improve the aesthetic look during use.

To achieve the aforementioned objective, the present disclosure provides a fragrance lanyard for hanging an article and being worn on a user's body, and the fragrance lanyard includes: a string, looped twice to form a loop with an overlapping part, provided for being worn and carried on a user's body, and including a hanging ring formed in a non-overlapping area of the string, and a string cap wrapped on two ends of the string separately for preventing the string from being frayed and for position limiting to prevent loosening; a pair of length adjusters, each having two lanyard holes formed adjacent to each other, and each length adjuster being designed with an 8-shaped cross section, such that the string can sequentially pass through the pair of length adjusters, and the pair of length adjusters can limit the overlapping part of the string to adjust the length of the string; a clasp, installed at the position of the hanging ring, and including a hanging hook corresponding to an article to be hung; a fragrance container, fixed to any one of the string caps or any one of the length adjusters or the clasp by a fixation means, and having a containing space defined in the fragrance container; and an aromatic element, arranged in the containing space, for making the fragrance container to emit fragrance. In an embodiment of the present disclosure, the fragrance container of the fragrance lanyard can be quickly assembled to any one of the string caps or any one of the length adjusters or the clasp by various fixation means, and these fixation means include: 1. The fragrance container has an end provided with an opening and another end provided with a round tube body having at least one through hole, and the outer periphery of the opening is provided with a first threaded part, and any one of the string caps or any one of the length adjusters or the clasp is provided with a corresponding second threaded part for fixing the fragrance container onto the second threaded part by a screwing method. 2. The fragrance container has an end provided with an opening and another end provided with a round tube body having at least one through hole, the outer periphery of the opening is provided with a first magnetic element, and any one of the string caps or any one of the length adjusters or the clasp is provided with a corresponding second magnetic element for fixing the fragrance container onto the second magnetic element by a magnetic attraction method. 3. The fragrance container has an end provided with an opening and another end provided with a round tube body having at least one through hole, and a cover corresponding to the opening is provided for sealing the opening, and any one of the string caps or any one of the length adjusters or the clasp is provided with a soft bushing having a diameter slightly smaller than the diameter of the fragrance container for tightly wrapping the fragrance container into the bushing by a socketing method for fixation.

In an embodiment of the present disclosure, a filter is provided in the through hole to serve as a protective means and provide a good ventilation effect. In addition, the aromatic element of the present disclosure is also available in many different forms: 1. The aromatic element of the present disclosure is a cotton rod provided for adsorbing an aromatic essential oil, and a fragrance is released slowly after the adsorption of a liquid that emitting fragrance such as the aromatic essential oil or a perfume through the cotton rod. 2. The aromatic element is formed by a plurality of aromatic balls or particles, and has a sweet aroma that can release fragrance slowly. In addition, the string of the present disclosure is formed by weaving a natural fiber, a synthetic fiber or their combination to maintain a specific aesthetic look and sturdiness and has a physical property that is tough and uneasily breakable.

In addition, the fragrance lanyard of the present disclosure further includes a hook arranged on the outer surface of the fragrance container, so that the fragrance container can be hung on articles other than the fragrance lanyard. In addition, the fragrance lanyard further includes a pendant arranged on the outer surface of the fragrance container.

### BRIEF DESCRIPTION OF THE DRAWINGS

FIG. 1 is a schematic view showing the structure of a preferred embodiment of the present disclosure;

FIG. 2 is a cross-sectional view of a preferred embodiment of the present disclosure, after installation.

FIG. 3 is a first schematic view showing a status of use of a preferred embodiment of the present disclosure;

FIG. 4 is a second schematic view showing a status of use of a preferred embodiment of the present disclosure; and

FIG. 5 is a third schematic view showing a status of use of a preferred embodiment of the present disclosure.

### DESCRIPTION OF THE PREFERRED EMBODIMENTS

To make it easier for the examiner to understand the content of the present disclosure, the specification accompanied by the drawings is described as follows.

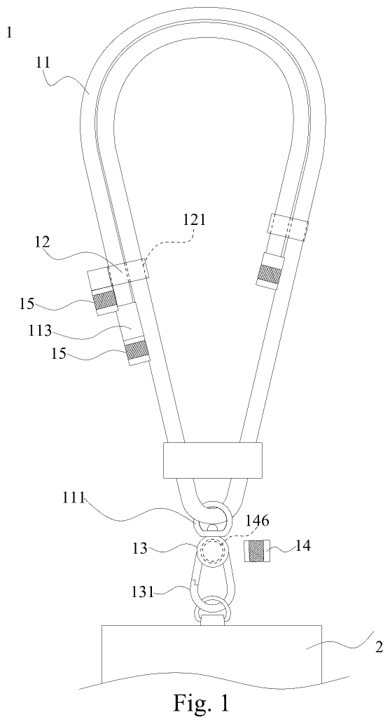

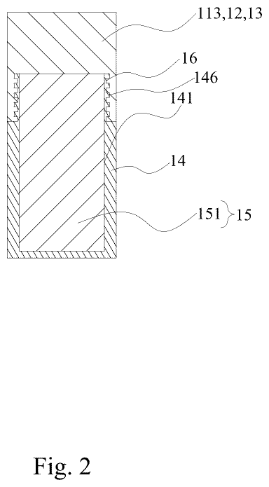

With reference to FIGS. 1, 2 and 3 to 5 for the schematic view of the structure, the cross-sectional view of the structure after installation, and the schematic views of different statuses of use in accordance with a preferred embodiment of the present disclosure respectively, the fragrance lanyard 1 of the present disclosure includes a string 11, a pair of length adjusters 12, a clasp 13, a fragrance container 14 and an aromatic element 15, and the fragrance lanyard is provided for hanging at least one article 2 (which is a mobile phone in this embodiment) and being worn on a user's body.

The string 11 is formed by weaving a natural fiber, a synthetic fiber or their combination, which has a physical property of tough and uneasily breakage, and the string 11 is looped twice to form a loop with an overlapping part and provided for being worn and carried on a user' body, and a non-overlapping area of the string 11 is formed with a hanging ring 111, and the string 11 has two end 112, wherein a string cap 113 is wrapped on each end 112 to prevent the string 11 from being frayed and for position limiting to prevent loosening.

Each length adjuster 12 has two lanyard holes 121 formed adjacent to each other, and each length adjuster is designed with a cross section in the 8-shape, such that the string 11 can sequentially pass through the pair of length adjusters 12, and the pair of length adjusters 12 can limit the overlapping part of the string 11 to adjust the length of the string 11.

The clasp 13 is arranged at the position of the hanging ring 111, and the clasp 13 is provided with a hanging hook 131 for hanging the article 2.

The fragrance container 14 is fixed to any one of the string caps 113 or any one of the length adjusters 12 or the clasp 13 by a fixation means, and the inside of the fragrance container 14 is defined with a containing space 141. In addition, the outer surface of the fragrance container 14 is provided with a hook 142 and a pendant 143, which can be hung and used for any article 2 other than the fragrance lanyard 1 to improve the aesthetic look during use.

The aromatic element 15 is arranged in the containing space 141, such that the fragrance container 14 emits fragrance. It is noteworthy that the aromatic element 15 of the present disclosure is a cotton rod 151 for adsorbing an aromatic essential oil, and a liquid with fragrance is released slowly after the adsorption of the aromatic essential oil or a perfume through the cotton rod. In addition, the aromatic element 15 is formed by a plurality of aromatic balls or particles, which has a sweet aroma and slowly releases a fragrance.

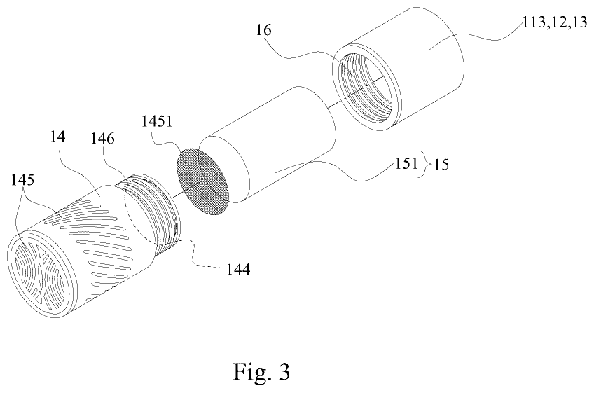

With reference to FIG. 3 for the schematic view showing the using status of the first fixation means of the present disclosure, the fragrance container 14 of the present disclosure has an opening 144 formed at an end of the fragrance container 14, a round tube body with at least one through hole 145 arranged at another end of the fragrance container 14, and the outer periphery of the opening 144 is formed with a first threaded part 146, and any one of the string caps 113 or any one of the length adjusters 12 or the clasp 13 is provided with a corresponding second threaded part 16 for fixing the fragrance container 14 onto the second threaded part 16 by a screwing method. It is noteworthy that a filter 1451 is provided in the through hole 145 to serve as a protective means and provide a good ventilation effect.

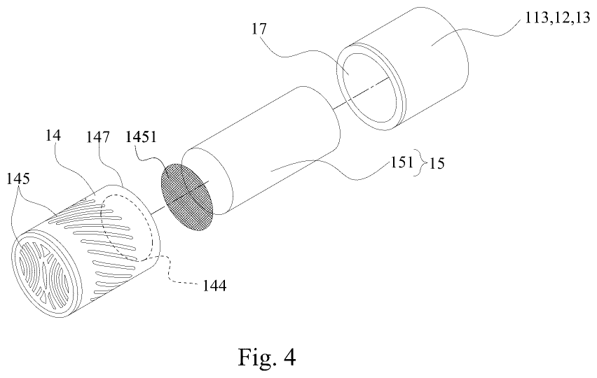

With reference to FIG. 4 for the schematic view showing the using status of the second fixation means of the present disclosure, the fragrance container 14 of the present disclosure has an opening 144 formed at an end of the fragrance container 14, a round tube body with at least one through hole 145 arranged at another end of the fragrance container 14, and the outer periphery of the opening 144 is provided with a first magnetic element 147, and any one of the string caps 113 or any one of the length adjusters 12 or the clasp 13 is provided with a corresponding second magnetic element 17 for fixing the fragrance container 14 onto the second magnetic element 17 by a magnetic attraction method.

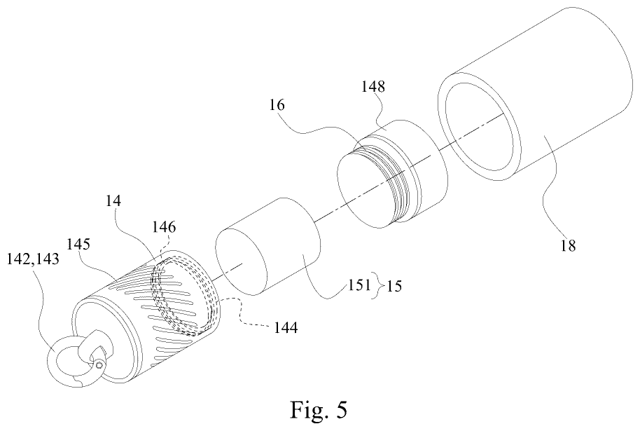

With reference to FIG. 5 for the schematic view showing the using status of the third fixation means of the present disclosure, the fragrance container 14 of the present disclosure has an opening 144 formed at an end of the fragrance container 14, a round tube body with at least one through hole 145 arranged at another end of the fragrance container 14, and a cover 148 corresponding to the opening 144 is provided for sealing the opening 144, and any one of the string caps 113 or any one of the length adjusters 12 or the clasp 13 is provided with a soft bushing 18, wherein the diameter of the bushing 18 is slightly smaller than the diameter of the fragrance container 14, so that the fragrance container 14 can be tightly wrapped into the busing 18 by a socketing method, wherein the bushing 18 is made of a material selected from rubber, silicone, or various soft material with elasticity.

In this way, the fragrance container 14 of the fragrance lanyard 1 of the present disclosure can be installed quickly to any one of the string caps 113 or any one of the length adjusters 12 or the clasp 13 by a fixation means for use alone. When it is desired to have a stronger smell of the lanyard, the two string caps 113 at both ends of the lanyard, the length adjusters 12, and even the clasp 13 can be installed with the fragrance container 14 at the same time, in order to emit a more concentrate fragrance. Therefore, the present disclosure can be assembled and used according to user needs, and the fragrance lanyard 1 can emit fragrance at any time when worn on the user's body by quickly assembling various types of the aromatic element 15 in the fragrance container 14. Further, the fragrance container 14 can also be directly made in any shape to further improve the aesthetic look during use and for other purposes.

What is claimed is:

1. A fragrance lanyard, provided for hanging an article and being worn on a user's body, comprising:
a string, looped twice to form a loop with an overlapping part, provided for being worn and carried on the user's body, and the string comprising a hanging ring formed in a non-overlapping area of the string, and a string cap wrapped on two ends of the string separately for preventing the string from being frayed and for position limiting to prevent loosening;
a pair of length adjusters, each having two lanyard holes formed adjacent to each other, and each of the pair of length adjusters being designed with an 8-shaped cross section, such that the string can sequentially pass through the pair of length adjusters, and the pair of length adjusters limits the overlapping part of the string to adjust a length of the string;
a clasp installed at a position of the hanging ring, and comprising a hanging hook corresponding to an article to be hung;
a fragrance container fixed to any one of the string caps, any one of the pair of length adjusters, or the clasp by a fixation means, and having a containing space defined in the fragrance container; and
an aromatic element arranged in the containing space for making the fragrance container to emit fragrance.

2. The fragrance lanyard according to claim 1, wherein the fragrance container has an end provided with an opening and another end provided with a round tube body having at least one through hole, an outer periphery of the opening is provided with a first threaded part, and any one of the string caps, any one of the pair of length adjusters, or the clasp is provided with a corresponding second threaded part for fixing the fragrance container onto the corresponding second threaded part by a screwing method.

3. The fragrance lanyard according to claim 1, wherein the fragrance container has an end provided with an opening and another end provided with a round tube body having at least one through hole, an outer periphery of the opening is provided with a first magnetic element, and any one of the string caps, any one of the pair of length adjusters, or the clasp is provided with a corresponding second magnetic element for fixing the fragrance container onto the corresponding second magnetic element by a magnetic attraction method.

4. The fragrance lanyard according to claim 1, wherein the fragrance container has an end provided with an opening and another end provided with a round tube body having at least one through hole, and a cover, corresponding to the opening, is provided for sealing the opening, and any one of the string caps, any one of the pair of length adjusters, or the clasp is provided with a soft bushing for tightly wrapping the fragrance container into the soft bushing by a socketing method.

5. The fragrance lanyard according to claim 2, wherein the at least one through hole is provided with a filter therein.

6. The fragrance lanyard according to claim 3, wherein the at least one through hole is provided with a filter therein.

7. The fragrance lanyard according to claim 4, wherein the at least one through hole is provided with a filter therein.

8. The fragrance lanyard according to claim 2, wherein the aromatic element is a cotton rod for adsorbing an aromatic essential oil, and a fragrance is released slowly after an adsorption of the aromatic essential oil or a perfume through the cotton rod.

9. The fragrance lanyard according to claim 3, wherein the aromatic element is a cotton rod for adsorbing an aromatic essential oil, and a fragrance is released slowly after an adsorption of the aromatic essential oil or a perfume through the cotton rod.

10. The fragrance lanyard according to claim 4, wherein the aromatic element is a cotton rod for adsorbing an aromatic essential oil, and a fragrance is released slowly after an adsorption of the aromatic essential oil or a perfume through the cotton rod.

11. The fragrance lanyard according to claim 2, wherein the aromatic element is formed by a plurality of aromatic balls or particles, which has a sweet aroma and slowly releases a fragrance.

12. The fragrance lanyard according to claim 3, wherein the aromatic element is formed by a plurality of aromatic balls or particles, which has a sweet aroma and slowly releases a fragrance.

13. The fragrance lanyard according to claim 4, wherein the aromatic element is formed by a plurality of aromatic balls or particles, which has a sweet aroma and slowly releases a fragrance.

14. The fragrance lanyard according to claim 1, wherein the string is formed by weaving one selected from the group of a natural fiber, a synthetic fiber, and a combination thereof, which has a physical property that is tough and uneasily breakable.

15. The fragrance lanyard according to claim 1, further comprising a hook arranged on an outer surface of the fragrance container.

16. The fragrance lanyard according to claim 1, further comprising a pendant arranged on an outer surface of the fragrance container.
